(19) United States
(12) Patent Application Publication
(10) Pub. No.: US 2022/0088202 A1
(43) Pub. Date: Mar. 24, 2022

(54) Nucleic Acid-Drug Conjugate, Drug Delivery System, Preparation Method Therefor and Use Thereof

(71) Applicant: Shanghai Jiaotong University, Shanghai (CN)

(72) Inventors: Chuan Zhang, Shanghai (CN); Jiao Zhang, Shanghai (CN); Yuanyuan Guo, Shanghai (CN)

(73) Assignee: Shanghai Jiaotong University, Shanghai (CN)

(21) Appl. No.: 17/424,954

(22) PCT Filed: Jan. 23, 2019
(86) PCT No.: PCT/CN2019/072790

§ 371 Date: Jul. 22, 2021

(51) Int. Cl. A61K 47/54; A61K 31/4745; A61K 31/337; C07H 21/02

(52) U.S. Cl. CPC A61K 47/549; C07H 21/02; A61K 31/337; A61K 31/4745

(57) ABSTRACT

Disclosed are a nucleic acid-drug conjugate based on a phosphorothioate-modified nucleic acid, a drug delivery system, and a preparation method therefor. The nucleic acid-drug conjugate is formed by reacting and conjugating a phosphorothioate group in a phosphorothioate-modified nucleic acid with a group that is modified on a drug molecule and able to undergo electrophilic reaction with the phosphorothioate groups; in addition, by selecting different nucleic acid sequences including functional nucleic acids, the nucleic acid-drug conjugate is able to be self-assembled into various forms of drug-containing nano-carriers for drug delivery.

### CROSS-REFERENCE TO RELATED APPLICATIONS

This application is a National Stage of PCT/CN2019/072790, filed Jan. 23, 2019, which is herein incorporated by reference in its entirety.

### INCORPORATION BY REFERENCE OF SEQUENCE LISTING

The instant application contains a Sequence listing which has been submitted in ASCII format via EFS-Web and is hereby incorporated by reference in its entirety. Said ASCII copy, created on Nov. 3, 2021, is named “431878-000013_ST25” and is about 2 KB in size.

### TECHNICAL FIELD

The present disclosure belongs to the field of biomedicine, and particularly relates to a nucleic acid-drug conjugate based on a phosphorothioate-modified nucleic acid, a drug delivery system, a preparation method therefor and use thereof.

### BACKGROUND ART

Chemotherapy is one of the important methods of tumor treatment. However, most chemotherapeutic drugs have defects such as poor water solubility, non-targeting, high blood clearance and even serious toxic side effects, resulting in their low bioavailability (Nat. Rev. Cancer 2006, 6, 789.), and long-term use will develop drug resistance, which brings certain limitations to their clinical applications.

In order to overcome this problem, in the past few decades, researchers have designed a series of nano drug delivery systems based on polymers or inorganic nanoparticles to improve the properties of chemotherapeutic drugs and promote their therapeutic effects, such as micelles, vesicles, liposomes, albumin nanoparticles and microvesicles (Science 2004, 303, 1818). Currently, the methods of nano-carrier encapsulating chemotherapy drugs mainly include physical embedding methods and chemical binding methods. However, due to the low drug loading and the easy leakage of drugs and the toxic side effects of the former, the drug conjugate strategy has become another hot spot in drug delivery. There are many materials used for drug conjugates, including polymers (J. Controlled Release 2016, 222, 116.), peptides (Adv. Drug Delivery Rev. 2017, 110-111, 112.), and antibodies (Trends Biotechnol. 2017, 35, 466.). However, the materials used to prepare drug conjugates often cannot meet the three characteristics of biocompatibility, biodegradability, and low immunogenicity at the same time, thus limiting their clinical translational application. In addition, some drug conjugates reported so far have the following problems: fewer drug graftings, uncontrollable drug grafting sites and grafting numbers, and limited drug molecules that can be grafted.

In recent years, nucleic acid, as a natural biopolymer, has attracted more and more attention in the field of biomedicine because it can meet the three conditions of biocompatibility, biodegradability and low immunogenicity at the same time, and has other specific molecular characteristics such as targeting, the specific molecular recognition function and the exact controllability of nanostructures formed by self-assembly.

At present, the studies of drug delivery using nucleic acid nanostructures (J. Polym. Sci. 2017, 35, 1.) such as polyhedrons and origami structures are also emerging in endlessly. For example, delivery of DOX is carried by using the characteristic that doxorubicin (DOX) can be inserted into the DNA double helix structure, and it has achieved good anti-tumor effect both in vivo and in vitro. However, the physical encapsulation of drugs by the nucleic acid insertion method is not only limited the drugs, but also their in vivo stability remains to be studied.

In addition, some researchers have inserted nucleic acids and chemotherapeutics into DNA nanostructures through functional groups modified at the ends of nucleic acid sequences or through polymers linked to DNA to achieve drug delivery. However, the former has relatively low drug loading due to the limitation of the number of functional groups, and the latter is able to cause certain biocompatibility problems due to the inclusion of polymer chain segments, and these two methods often require relatively complicated synthetic processes.

Therefore, it is urgent to establish a novel biocompatible nucleic acid nano drug-loaded system which is universally applicable to chemotherapeutic drugs and can precisely control the drug loading and drug loading sites.

### SUMMARY

The first objective of the present disclosure is to provide a nucleic acid-drug conjugate based on a phosphorothioate-modified nucleic acid to achieve accurate and controllable grafting and efficient delivery of chemotherapeutic drugs, and solve the following shortcomings of the current delivery system of drug conjugate: (1) the three requirements of biocompatibility, biodegradability and low immunogenicity of the carrier material cannot be met at the same time; (2) it is difficult to precisely control the drug grafting site and drug loading; (3) there are restrictions on the drugs that can be grafted, and the universality of nucleic acid as a carrier material cannot be achieved, and it is difficult to reduce the cost of nucleic acid drug-loaded; (4) a relatively complicated synthetic process is required; and (5) a reasonable and simple construction of a multifunctional drug-loaded system cannot be achieved.

The second objective of the present disclosure is to provide a drug delivery system, which is a nano drug-loaded system formed by self-assembly of the above nucleic acid-drug conjugate.

The third objective of the present disclosure is to provide a method for preparing the nucleic acid-drug conjugate based on a phosphorothioate-modified nucleic acid.

The fourth objective of the present disclosure is to provide a method for preparing the above drug delivery system.

The fifth objective of the present disclosure is to provide a use of the above nucleic acid-drug conjugate and the drug delivery system in preparation of tumor therapeutic drugs by using nucleic acid as a carrier.

The sixth objective of the present disclosure is to provide a drug, including the drug delivery system formed by the above nucleic acid-drug conjugate.

The technical scheme of the present disclosure is as follows:

A nucleic acid-drug conjugate based on a phosphorothioate-modified nucleic acid, comprising a phosphorothioate-modified nucleic acid skeleton and a drug molecule grafted onto the nucleic acid skeleton, wherein, the grafting is realized by reacting the phosphorothioate group on the nucleic acid skeleton with a group modified on the drug molecule that is able to undergo electrophilic reaction with the phosphorothioate group. A functional nucleic acid-drug conjugate (i.e, a nucleic acid-drug conjugate) is formed from the nucleic acid skeleton and the drug molecule, and the nucleic acid-drug conjugate can be self-assembled to obtain a nano drug-loaded system.

In some embodiments, on the phosphorothioate-modified nucleic acid skeleton, the sites and number of phosphorothioate modifications are able to be adjusted and controlled as required, and the phosphorothioate is continuously modified at one end of the nucleic acid sequence, and/or is selectively modified at the middle base sequence of the nucleic acid sequence, and the modification mode is multiple modification or single modification.

In some embodiments, the sites and number of phosphorothioate modifications are able to be adjusted by preparing phosphorothioate-modified nucleic acid by a solid-phase synthesis method.

In some embodiments, the sequence and segment type of the oligonucleotide of the phosphorothioate-modified nucleic acid skeleton are able to be independently designed, a controllable DNA nanostructure is able to be further assembled through molecular recognition, and the nucleic acid-drug conjugate and the assembled structure thereof is able to be used as a novel drug delivery system to prepare controllable drug delivery system.

In some embodiments, the nucleic acid skeleton may be designed with different sites and numbers of phosphorothioate modifications according to different requirements, for example, for the nucleic acids used for assembly of gel and tetrahedral structure, considering the steric hindrance after modification and its effect on base pairing, a phosphorothioate modification site is set every 2 to 3 bases on the nucleic acid skeleton; while the nucleic acids used for micellar assembly, considering the micellar assembly mechanism, tend to carry out continuous phosphorothioate modifications at one end of a nucleic acid sequence to prepare block-type nucleic acids containing phosphodiester bonds and phosphorothioate bonds.

In some embodiments, a group that is able to undergo electrophilic reaction with phosphorothioate is introduced into the drug molecule by a simple esterification or acylation reaction.

In some embodiments, a cleavable responsive chemical bond is also introduced into the drug molecule, the cleavable responsive chemical bond may include disulfide bond, acylhydrazone bond, ester bond, but is not limited to the responsive chemical bond listed above.

In some embodiments, the group that is able to modified on the drug molecule and undergo electrophilic reaction with phosphorothioate is selected from one or more of the following: 1) a bromine-containing or iodine-containing functional group such as iodo or bromo acetyl compounds, γ-bromo-α,β-unsaturated carbonyl, benzyl bromide or bromomaleimide; 2) a maleimide group; 3) an aziridinyl sulfonamide group, but not limited to the above.

In some embodiments, the drug molecules are selected from anticancer drugs (such as paclitaxel, camptothecin, cisplatin, docetaxel, chlorambucil, methotrexate, doxorubicin, cisplatin predrug, but not limited to the above) and cancer-targeted drug molecules (such as erlotinib, imatinib, gefitinib, sorafenib, but not limited to the above listed).

In some embodiments, the drug molecules are bromo-modified drug molecules containing a disulfide bond. For example, the carbonyl ethyl bromide structure and benzyl bromide structure may be introduced by simple chemical reactions to realize further reaction with the nucleic acid skeleton modified by the phosphorothioate skeleton, and the introduction of disulfide bonds in this process may also realize the redox release of the drug. However, this process is not limited to carbonyl ethyl bromide structure and benzyl bromide structures, other compounds that able to undergo electrophilic reaction with phosphorothioate also may be introduced; and this structure is not limited to the introduction of disulfide bonds, other types of cleavable bonds may also be used to replace disulfide bonds, such as ester bonds that able to be broken by esterase, chemical bonds that able to be broken by light or by acid.

In some embodiments, the drug molecules grafted on the nucleic acid skeleton include functional drug molecules, fluorescent probe molecules, and cell targeting molecules.

The drug molecules are anti-tumor drugs. However, the drug molecules of the present disclosure are not limited to anti-tumor drugs, drugs for the treatment of other diseases or drug molecules for imaging may also be used in this way to achieve nucleic acid modification; in some specific embodiments, the anti-tumor drug is camptothecin or paclitaxel. However, the anti-tumor drugs of the present disclosure are not limited to camptothecin and paclitaxel. Other modifiable drugs, such as cisplatin, docetaxel, chlorambucil, methotrexate, doxorubicin, may also be used.

In the present disclosure, there are no limitation on the type and sequence of the nucleic acid skeleton. In terms of species, both deoxyribonucleic acid sequences and ribonucleic acid sequences can be selected. In terms of sequence requirements, non-functional common base sequences can be selected, including simple nucleic acid sequences composed of one base and complex nucleic acid sequences that is able to be used for precise structure assembly of nucleic acids; functional nucleic acid sequences can also be selected, the functional nucleic acid sequences is selected from antisense nucleic acid sequence, nucleic acid aptamer sequence, nuclease sequence, small interfering RNA, messenger RNA, micro RNA, long non-coding RNA, small hairpin RNA, guide RNA for gene editing, and circular RNA.

In the present disclosure, the nucleic acid molecule grafted with the drug retains the property of base complementary pairing, by which the other functional nucleic acid sequences are paired to impart targeting and imaging functions to the nucleic acid-drug conjugate drug delivery system to prepare a multifunctional drug delivery system of nucleic acid-drug conjugate, wherein the functional nucleic acid for pairing is selected from nucleic acid aptamers, antisense nucleic acid sequences, fluorescent molecule-modified nucleic acid sequences, functional polypeptide-modified nucleic acid sequences, and targeted galactose-modified nucleic acid sequences.

The present disclosure also provides a drug delivery system, which is a nano drug-loaded system formed by self-assembly of the above nucleic acid-drug conjugate.

In the present disclosure, there are no limitation on the form of the assembly prepared by using the above nucleic acid-drug conjugate, for example:

Simple prodrugs of the nucleic acid-drug macromolecular may be prepared by design; the design method may be as following: reducing the number of phosphorothioates or selecting small molecules with strong hydrophilicity.

Precisely assembled DNA nanostructures are able to be designed, such as drug-loaded nucleic acid polyhedral structures and not limited to DNA tetrahedrons, DNA (but not limited to) origami structures of different sizes may also be constructed; RNA nanostructures can also be selected. The design method could be as follows: selecting a DNA or RNA sequence with a specific sequence, carrying out phosphorothioate modification at a specific position on the nucleic acid skeleton, and performing assembly through base complementary pairing after being conjugated with drugs.

DNA nanogels may be prepared by design; the design method could be as follows: selecting a DNA or RNA sequence with a specific sequence by selecting a DNA or RNA sequence with a special sequence, preparing a Y-type or i-type assembly body by carrying out phosphorothioate modifications at specific positions on the nucleic acid skeleton, and then further assembling to prepare a drug-loaded nanogels.

The drug-loaded micellar spherical nucleic acid may be prepared by design; the design method could be as follows: selecting common or functional nucleic acid sequence, continuously carrying out phosphorothioate modifications at one end to prepare a block-type nucleic acid with a phosphodiester structure at one end and a phosphorothioate structure at the other end, and carrying out drug modification at the phosphorothioate modification site; since the hydrophilic shell of the micellar spherical nucleic acid retains a property of base complementary pairing, the micellar spherical nucleic acid is endowed with the function of targeting and imaging through this property;

Drug-loaded nucleic acid hydrogel.

In some embodiments, the drug molecules used to prepare precisely assembled DNA nanostructures are selected from molecules with weak hydrophobicity and small molecular weight, and/or molecules with reduced number of phosphorothioate modifications on the nucleic acid skeleton; and the drug molecules used for micellar assembly are selected from highly hydrophobic molecules, and/or molecules with increased number of phosphorothioate modifications on the nucleic acid skeleton.

In another aspect, the present disclosure provides a method for preparing a nucleic acid-drug conjugate based on a phosphorothioate-modified nucleic acid, comprising the following steps:

Step one, preparing phosphorothioate-modified nucleic acid molecules; preferably, the phosphorothioate-modified nucleic acid molecule is prepared by solid-phase synthesis, so that the site and number of phosphorothioate modifications are able to be adjusted;

Preparing drug molecules containing groups that are able to undergo electrophilic reaction with phosphorothioate by a chemical reaction method; in some preferred embodiments, the drug molecule is further introduced with a cleavable responsive chemical bond, more preferably, the cleavable responsive chemical bond is a disulfide bond, and the group that undergo electrophilic reaction with phosphorothioate is a bromo-modified group; the chemical reaction method for preparing a bromo-modified drug molecule containing disulfide bonds is selected from one of the following methods: introducing the disulfide bond into the camptothecin drug molecule through triphosgene under argon atmosphere, and then reacting with bromoacetyl bromide to obtain the camptothecin bromide drug molecule; and, carrying out an esterification reaction of 4-bromomethylbenzyl alcohol with dithiodipropylene to prepare the carboxylic acid structure containing disulfide bonds, and then carrying out an esterification reaction of the carboxylic acid with the hydroxyl group of paclitaxel to prepare the benzyl bromide structure-modified paclitaxel drug molecule.

There is no order among the steps of preparing nucleic acid molecules and preparing drug molecules;

Step two, preparing a nucleic acid-drug conjugate, dissolving the modified drug molecule in an organic solvent, then adding an appropriate amount of nucleic acid molecule for the reaction, the drug molecule in the reaction is greatly excessive relative to the phosphorothioate group, removing the excess small molecules after reaction, and drying to obtain the nucleic acid-drug conjugate. The treatment process after the reaction can be as follows: adding a certain volume of aqueous solution (the volume of the aqueous solution may be determined through limited number of experiments, which will not be described in detail herein), then removing excess small molecules by ethyl acetate extraction or ethanol precipitation, and drying the water phase to obtain the nucleic acid-drug conjugate.

Preferably, in the above preparation method, the grafting efficiency of drug molecules is able to be controlled by controlling the concentration of nucleic acid molecules, the ratio of drug molecules to phosphorothioate, and whether a salt solution is included in the reaction solution.

In some embodiments, the organic solvent is selected from one of the following: the reaction system used for grafting camptothecin is a mixed system of dimethyl sulfoxide and phosphate buffer, with a volume ratio of 4:1; and the reaction system used for grafting camptothecin is dimethyl sulfoxide.

In some embodiments, the temperature and time of the reaction are: reacting overnight or longer at 50° C.-58° C., and the reaction temperature and time may be changed and are not limited thereto.

In another aspect, the present disclosure provides a method for preparing the drug delivery system of the above nucleic acid-drug conjugate, wherein it is selected from one of the following methods:

Using a direct dissolution method, directly dissolving the nucleic acid-drug conjugate in an aqueous solution or a salt solution to prepare a prodrug of the drug-loaded nucleic acid macromolecular.

Preparation of drug-loaded nucleic acid polyhedral structure or nucleic acid gel: selecting nucleic acid sequences that able to be complementary paired, carrying out phosphorothioate modification at the characteristic sites to obtain the nucleic acid-drug conjugate, mixing the complementary paired nucleic acid sequences in a TAE/Mg²⁺ solution in proportion and preparing by an annealing; taking camptothecin as an example: mixing nucleic acid-camptothecin prodrugs with 4 different sequences in an equimolar amount in 1×TAE/Mg²⁺ solution buffer, placing them at 90° C. for 5 min and then quickly cooling to 4° C. to obtain the camptothecin-loaded DNA nano-tetrahedrons.

Preparation of drug-loaded micellar spherical nucleic acid: selecting a block-type nucleic acid modified by a segment of continuous phosphodiester bond and a segment of continuous phosphorothioate bond, preparing the nucleic acid-drug conjugate according to the above method, using a dialysis method to prepare the drug-loaded micelle, wherein the nucleic acid block that has not been modified by phosphorothioate is used as a hydrophilic shell of the micelle, and the hydrophobic drug is used as a core; specifically, taking paclitaxel as an example: dissolving the obtained block-type nucleic acid-paclitaxel conjugate in dimethyl sulfoxide, adding an equal volume of water, and then dialyzing the mixture in water overnight to obtain the paclitaxel-loaded micellar spherical nucleic acid.

Preparation of drug-loaded multifunctional spherical nucleic acid: selecting the nucleic acid sequence containing a complementary pairing part with a continuous phosphodiester bond block, where the nucleic acid sequence has the functions of fluorescent molecule modification, targeted aptamer modification, targeted polypeptide modification, and targeted small molecule modification; carrying out annealing on the micelles prepared in (3) to prepare the multifunctional drug-loaded spherical nucleic acid with targeting, imaging, gene therapy and chemotherapy. Specifically, for example, selecting a functional nucleic acid sequence (targeting or fluorescent modification) containing a complementary pairing part of the phosphodiester bond block in the spherical nucleic acid to prepare the multifunctional drug-loaded spherical nucleic acid with targeting, imaging, gene therapy and chemotherapy by annealing.

In another aspect, the present disclosure provides a use of the nucleic acid-drug conjugate and drug delivery system based on the above in preparation of nucleic acid nano-drugs and chemotherapeutic drugs based on gene therapy and chemotherapy combined treatment of diseases.

In another aspect, the present disclosure provides a drug, including the above nucleic acid-drug conjugate or a drug delivery system formed thereof.

In some embodiments, the drug is a tumor therapeutic drug.

The benefits of the present disclosure are as follows:

1. Nucleic acid-drug conjugates are obtained by grafting drug molecules on phosphorothioate-modified nucleic acid molecules, and the drug delivery system is obtained by self-assembly of nucleic acid-drug conjugates, so that the purpose of delivering drugs by using nucleic acid is realized, the drug delivery by using nucleic acid is successfully achieved, and the three major requirements of carrier material biocompatibility, in vivo degradability and low immunogenicity are met.

2. The phosphorothioate-modified oligonucleotides are obtained by solid-phase synthesis, without cumbersome chemical synthesis, the sites and number of phosphorothioate modifications are able to be adjusted and controlled as required, so that the purpose of precise design for the drug loading amount and drug loading sites in the later stage are achieve.

3. There are fewer restrictions on the drugs that can be grafted, and the universality of nucleic acid as a carrier material is realized.

4. The synthesis process is simple.

5. The required DNA nanostructure is able to be obtained by independently designing the sequence and segment type of oligonucleotides, thereby preparing a controllable drug delivery system.

6. The drug-loaded system uses biocompatible nucleic acid as a material, which has lower immunogenicity to the organism, lower metabolic burden, and no toxic side effects.

7. This drug-loaded system may further introduce targeting groups and functional nucleic acid sequences to prepare a multifunctional drug-loaded system of nucleic acid, so as to achieve the purpose of combined gene therapy and chemotherapy; and a DNA nanostructure integrated with tumor imaging may also be achieved.

8. The reasonable and simple construction of a multifunctional drug-loaded system is realized.

Of course, not all the advantages described above need be achieved for any product implementing the present disclosure at the same time.

### BRIEF DESCRIPTION OF THE DRAWINGS

FIG. 1 shows the synthetic route of the bromocamptothecin prodrugs and bromopaclitaxel molecules in Example 1 and Example 2.

FIG. 2 shows an ¹H NMR spectrum of the prodrug compound 1 in Example 1.

FIG. 3 shows a LC-MS spectrum of the prodrug compound 1 in Example 1.

FIG. 4 shows an ¹H NMR spectrum of the prodrug compound 2 in Example 1.

FIG. 5 shows a LC-MS spectrum of the prodrug compound 2 in Example 1.

FIG. 6 shows a UV-Vis spectrophotometric spectrum of the camptothecin-modified oligonucleotide prodrug in Example 1.

FIG. 7 shows a denaturing gel electrophoresis spectrum of the camptothecin-modified oligonucleotide prodrug in Example 1.

FIG. 8 shows a matrix-assisted laser desorption ionization time of flight mass spectrometry (MALDI-TOF MS) of the camptothecin-modified oligonucleotide prodrug in Example 1.

FIG. 9 shows an ¹H NMR spectrum of DTDP-Bz-Br in Example 2.

FIG. 10 shows an ¹H NMR spectrum of PTX-Bz-Br in Example 2.

FIG. 11 shows a mass spectrum of PTX-Bz-Br in Example 2.

FIG. 12 shows a ³¹P NMR spectrum of block type nucleic acid-paclitaxel molecule (DNA-b-PTX-g-DNA) in Example 2.

FIG. 13 shows a matrix-assisted laser desorption ionization time of flight mass spectrometry of DNA-b-PTX-g-DNA in Example 2.

FIG. 14 shows an agarose gel electrophoresis diagram of the camptothecin-modified DNA tetrahedral origami structure in Example 3.

FIG. 15 shows the hydrodynamic diameter data of the camptothecin-modified DNA tetrahedral origami structure in Example 3.

FIG. 16 is an atomic force microscope photograph showing the camptothecin-modified DNA tetrahedral origami structure in Example 3.

FIG. 17 is a schematic diagram showing the toxicity evaluation of the camptothecin-modified DNA tetrahedral origami structure on cancer cells in Example 3.

FIG. 18 is a schematic diagram showing the cancer cell apoptosis achieved by the camptothecin-modified DNA tetrahedral origami structure in Example 3.

FIG. 19 shows a 20% deformed gel electrophoresis diagram of PTX-SNA in Example 4.

FIG. 20 shows a 1% agarose gel electrophoresis diagram and dynamic light scattering diagram of PTX-SNA in Example 4.

FIG. 21 shows a transmission electron micrograph of PTX-SNA in Example 4.

FIG. 22 shows a 1% agarose gel electrophoresis diagram of FAM/AS1411/PTX-SNA of the multifunctional spherical nucleic acid in Example 4.

FIG. 23 shows an agarose gel electrophoresis diagram of the spherical nucleic acid AS1411/Bcl-2-PTX-SNA co-existing with targeted gene and chemotherapy in Example 4.

FIG. 24 is an graph showing in vitro anti-tumor and reversal of tumor multidrug resistance of various micellar spherical nucleic acids loaded with paclitaxel in Example 4.

### DETAILED DESCRIPTION OF THE EMBODIMENTS

The present disclosure provides a nucleic acid-drug conjugate based on a phosphorothioate-modified nucleic acid, a drug delivery system, a preparation method therefor and application thereof.

The present disclosure belongs to the field of biomedicine, and specifically discloses a nucleic acid-drug conjugate based on a phosphorothioate-modified nucleic acid, a drug delivery system, and their preparation method. The nucleic acid-drug conjugate is formed by grafting the phosphorothioate in the nucleic acid phosphorothioate skeleton with a drug molecule modified by an electrophilic reactive group that can react with the phosphorothioate, wherein, nucleic acid sequences including functional nucleic acids may be selected by different targets. In addition, the nucleic acid-drug conjugates are able to be self-assembled into drug-containing nano-carriers for drug delivery. Compared with the prior art, the phosphorothioate nucleic acid skeleton used in the present disclosure is able to be obtained by simple solid-phase synthesis. The grafting sites and assembly behavior of drug molecules on the nucleic acid skeleton may be precisely controlled by drug grafting with phosphorothioate groups. This method has universal applicability to chemotherapeutic drugs. According to the present disclosure, biocompatible and in vivo biodegradable water-soluble nucleic acid macromolecules are used as a carrier, which can significantly improve the physical and chemical properties and in vivo distribution properties of chemotherapeutic drugs, thus promoting their therapeutic effects. The combined treatment of gene therapy and chemotherapy may also be achieved, and the complex synthesis and modification steps are avoided.

In the present disclosure, the range represented by “one value to another value” is a general way to avoid listing all the values in the range one by one in the specification. Therefore, the record of a specific numerical range covers any numerical value within the numerical range and the smaller numerical range defined by any numerical value in the numerical range, as if the arbitrary numerical value and the smaller numerical value are clearly written in the specification.

The present disclosure will be further described below in conjunction with specific embodiments. It should be understood that these embodiments are only used to illustrate the present disclosure and not to limit the protection scope of the present disclosure. In practical applications, improvements and adjustments made by those skilled in the art according to the present disclosure still fall into the protection scope of the present disclosure.

### EXAMPLE 1

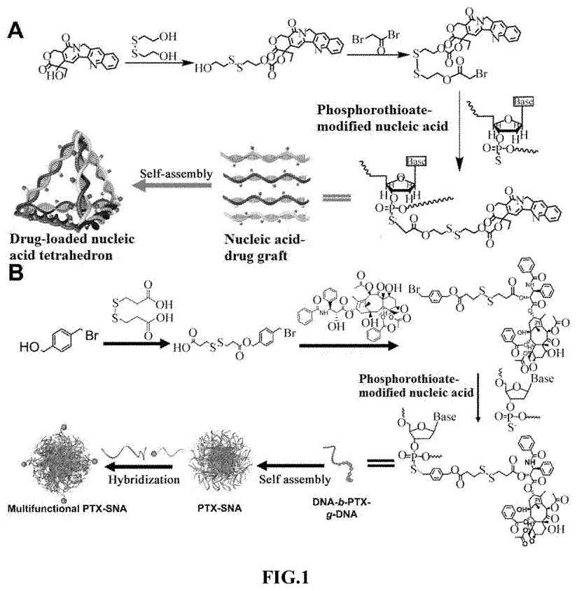

1.1 Synthesis of bromocamptothecin prodrugs, the steps were shown in FIG. 1 (A), the synthesis was carried out in two steps:

(1) Synthesis of redox-sensitive prodrug compound 1: camptothecin (1 g) and triphosgene (313 mg) were dissolved in 150 mL of anhydrous dichloromethane under argon atmosphere, 4-dimethylaminopyridine (DMAP, 1.12 g dissolved in 20 mL of dichloromethane) was slowly added dropwise, the mixture was stirred and reacted at room temperature for half an hour, then 2,2′-dithiodiethanol (4.43 g) was added thereto, and the mixture was reacted at room temperature overnight.

After the reaction, the mixed solution was washed with 80 mL of 0.1 M HCL solution, the resulting solution was layered, and the supernatant was discarded. After repeated washing with the HCL solution three times, 80 mL of saturated NaCl solution was used for washing, the resulting solution was layered, and the supernatant was discarded; 80 mL of distilled water was used for washing, the resulting solution was layered, then the supernatant was discarded, the resulting product was dried with anhydrous MgSO₄ to obtain a crude product, the crude product was separated and purified by column chromatography gradient elution, and the separation polarity was methanol:dichloromethane=1:100.

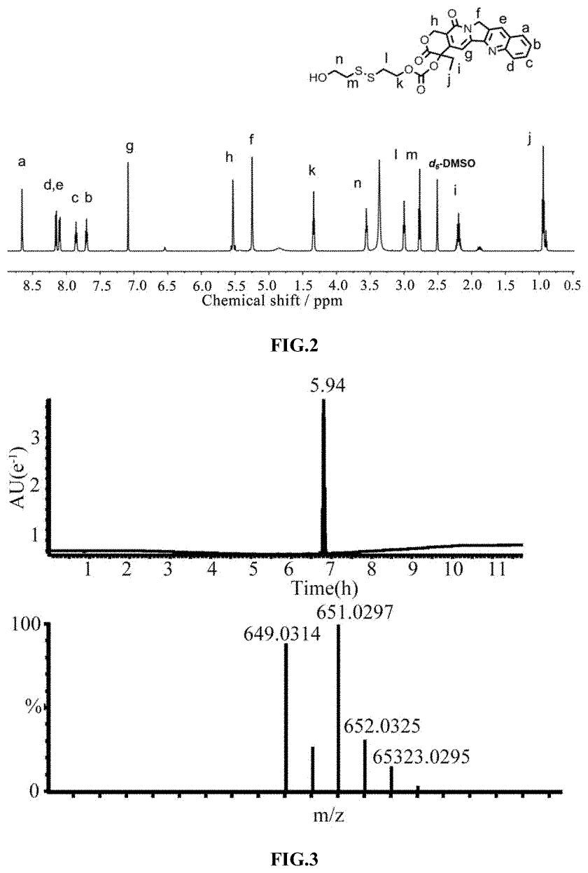

The yield of the product in this step was 64.5%, the NMR spectrum of the product was shown in FIG. 2. The peaks were attributed to: ¹H NMR (400 MHz, d₆-DMSO) δ (ppm): 8.67 (s, 1H), 8.15 (d, J=8.6 Hz, 1H), 8.11 (d, J=7.9 Hz, 1H), 7.85 (ddd, J=8.4, 6.9, 1.3 Hz, 1H), 7.70 (dd, J=11.1, 3.9 Hz, 1H), 7.03 (s, 1H), 5.56-5.38 (m, 2H), 5.34-5.19 (m, 2H), 4.38-4.19 (m, 2H), 3.62-3.46 (m, 2H), 3.02-2.88 (m, 2H), 2.80-2.64 (m, 2H), 2.30-2.01 (m, 2H), 0.98-0.84 (m, 3H). ¹³C NMR (400 MHz, d₆-DMSO) δ (ppm): 167.54, 156.93, 153.29, 152.62, 148.33, 146.69, 145.22, 132.00, 130.86, 130.19, 129.45, 128.94, 128.40, 128.14, 119.58, 94.86, 78.36, 66.92, 59.75, 50.70, 41.56, 36.63, 30.78, 8.05. The molecular weight of compound 1 measured by LC-MS liquid chromatography mass spectrometry was consistent with the theoretical value, as shown in FIG. 3, the calculated value of ESI-MS m/z=(M+H⁺) was 529.106, and the detected value of m/z=(M+H⁺) was 529.103.

(2) Synthesis of bromocamptothecin prodrug compound 2: compound 1 (330 mg) and N, N-diisopropylethylamine (88.8 mg) were dissolved in 150 mL of anhydrous dichloromethane under argon atmosphere, the solution was stirred, then bromoacetyl bromide (126.1 mg, dissolved in ultra-dry dichloromethane) was slowly added dropwise and reacted at room temperature overnight. After the reaction was completed, the solvent was evaporated to dryness using a rotary evaporator, and the product was purified by column chromatography gradient elution. Silica was used as the filler for column chromatography, and the separation polarity was methanol: dichloromethane=1:200.

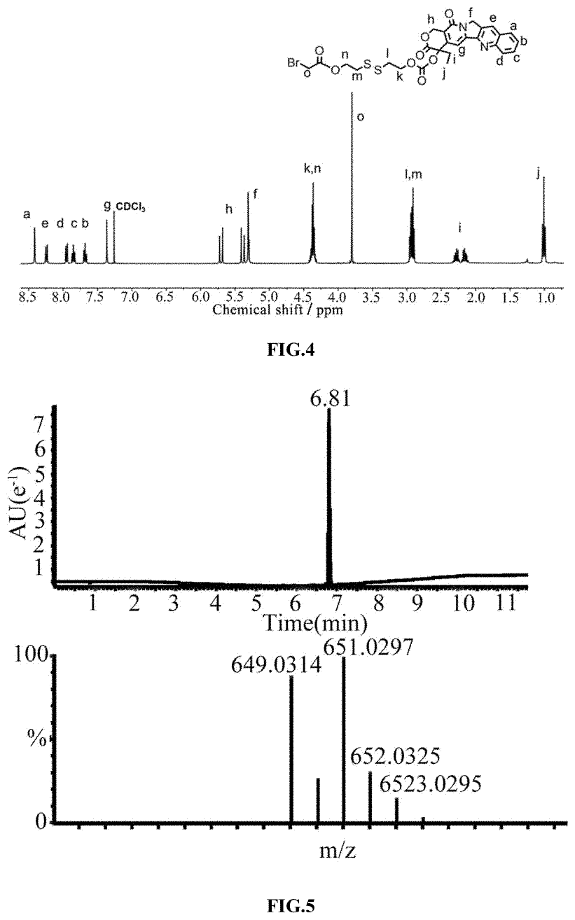

The yield of the product in this step was 80.7%, the NMR spectrum of the product was shown in FIG. 4. The peaks were attributed to: ¹H NMR (400 MHz, CDCl₃) δ (ppm): 8.41 (d, J=13.0 Hz, 1H), 8.21 (d, J=8.5 Hz, 1H), 7.91 (t, J=10.3 Hz, 1H), 7.90-7.71 (m, 1H), 7.66 (ddd, J=8.0, 7.0, 1.0 Hz, 1H), 7.33 (s, 1H), 5.48 (dt, J=27.5, 13.8 Hz, 2H), 5.31-5.22 (m, 2H), 4.48-4.20 (m, 4H), 3.87-3.72 (m, 2H), 3.01-2.80 (m, 4H), 2.38-2.04 (m, 2H), 0.99 (t, J=7.5 Hz, 3H). ¹³C NMR (400 MHz, CDCl₃) δ (ppm): 167.27, 166.98, 157.24, 153.44, 152.18, 148.71, 146.40, 145.57, 131.35, 130.82, 129.51, 128.50, 128.25, 128.18, 128.01, 120.24, 96.06, 78.06, 67.06, 66.48, 63.76, 53.50, 36.66, 31.85, 29.93, 25.66, 7.67. The molecular weight of compound 2 measured by LC-MS liquid chromatography mass spectrometry was m/z (M+H⁺)=649.031, which was consistent with the theoretical value m/z (M+H⁺) of 649.530, as shown in FIG. 5.

1.2 Synthesis of the prodrugs of oligonucleotide-camptothecin conjugate

In Example 1.2, four oligonucleotide sequences with complementary bases were selected, and the phosphorothioate modification sites were in the middle of the skeleton, and the number of phosphorothioate groups was: 7 for TET-A, 7 for TET-C; 8 for TET-B, and 8 for TET-D. The sequences of the four oligonucleotides were as follows:

TET-A:
(SEQ ID NO. 1)
5′-ACATTC*CTAAG*TCTGAAACATTAC*AGCT*TGCT*ACACGAGAAG
AGC*CGCC*ATAGTA-3′;
TET-B:
(SEQ ID NO. 2)
5′-TATCA*CCAG*GCAG*TTGACAGTGTAGC*AAGC*TGTAATAGATGC
G*AGGG*TCCA*ATAC-3′;
TET-C:
(SEQ ID NO. 3)
5′-TCAACTG*CCTG*GTGATAAAACGACAC*TACG*ACTA*TGGC*GGC
T*CTTC-3′;
TET-D:
(SEQ ID NO. 4)
5′-TTCAG*ACTT*AGGA*ATGTGCTTCCC*ACGT*AGTG*TCGTTTGTA
TTGG*ACCC*TCGCAT-3′;
(* represents the modification sites of the
phosphorothioate group).

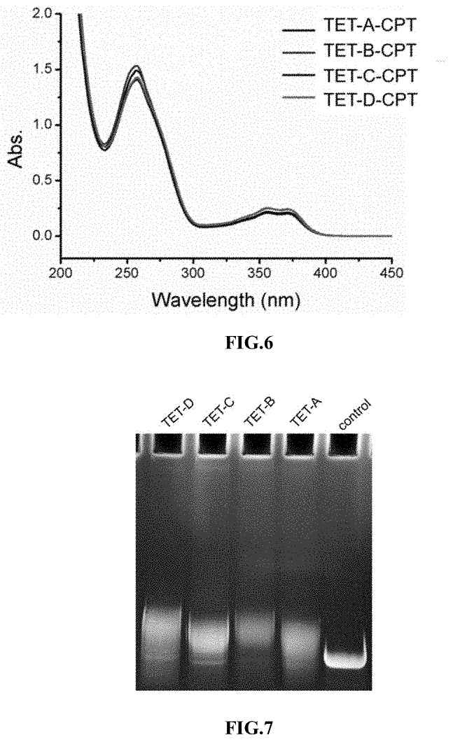

Bromocamptothecin prodrug compound 2 was dissolved in 80 μL of dimethyl sulfoxide solution, 20 μL of phosphate buffer of phosphorothioate-modified oligonucleotide was added, the oligonucleotide concentration was 350 μM (the ratio of the phosphorothioate group to the compound 2 was 1:50), the solution was placed at 55° C. for shaking for 20 h. After the reaction, the excess compound 2 in the reaction was extracted with ethyl acetate multiple times, the resulting product was re-dissolved in ultrapure water after being evaporated to dryness to obtain the camptothecin-modified oligonucleotide prodrug. The obtained oligonucleotide prodrug solutions with 4 kinds of base complementary camptothecin-modified were detected by a UV spectrophotometer. In addition to the characteristic absorption of DNA at 260 nm, the characteristic absorption peak of camptothecin molecule was appeared at 365 nm, as shown in the FIG. 6.

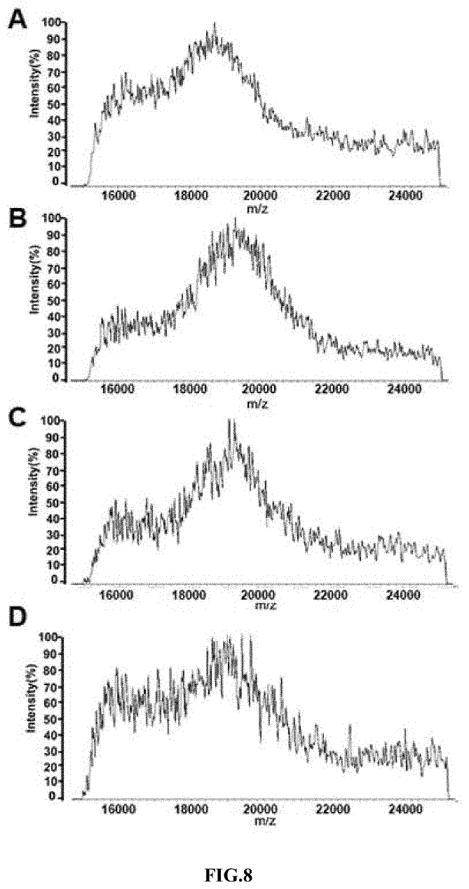

In this step, 2-6 different numbers of camptothecin molecules were grafted into each oligonucleotide chain, most of the oligonucleotides were grafted with 4 camptothecin molecules, the grafted number of TET-A and TET-C were less than those of TET-B and TET-D, the 20% denaturing polyacrylamide gel electrophoresis (PAGE) detection analysis was shown in FIG. 7 and the matrix-assisted laser desorption ionization time of flight mass spectrometry detection was shown in FIG. 8.

### EXAMPLE 2

### Synthesis of Nucleic Acid-Paclitaxel Graft

2.1 Synthesis of benzyl bromide modified paclitaxel drug (PTX-Bz-Br), the steps were shown in FIG. 1 (B), and the synthesis was also carried out in two steps:

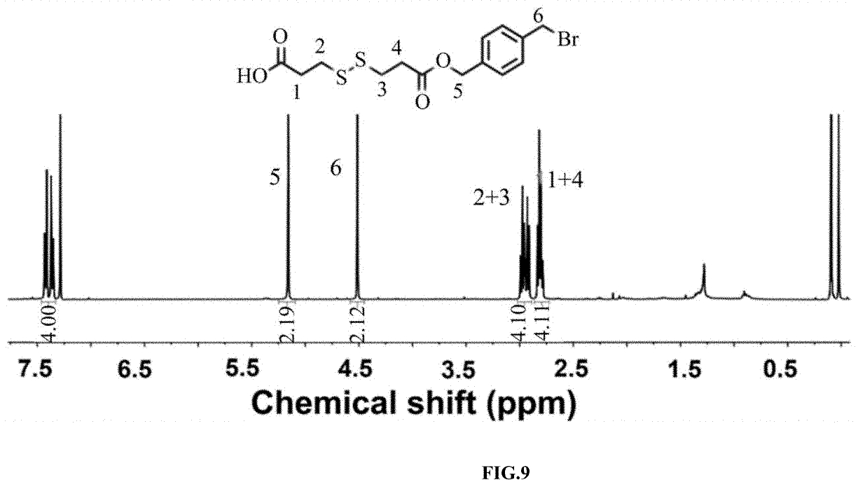

(1) 4-bromomethylbenzyl alcohol (500 mg, 1 equivalent) and dithiodipropionic acid (DTDP, 2.6 g, 5 equivalents) were dissolved in a mixed solution of ultra-dry dichloromethane and tetrahydrofuran (1/1, v/v); then DMAP (91 mg, 0.3 equivalent) was added thereto, the mixture was stirred for a few minutes and then dicyclohexylcarbodiimide (DCC, 615 mg, 1.2 equivalent, dissolved in ultra-dry dichloromethane) was added dropwise, after reacting at room temperature overnight, the solvent was evaporated to dryness using a rotary evaporator, and the disulfide bond-containing benzyl bromide structure (DTPA-Bz-Br) was separated by silica gel column chromatography, the eluent was petroleum ether/ethyl acetate. The NMR spectrum and attribution of the product were shown in FIG. 9.

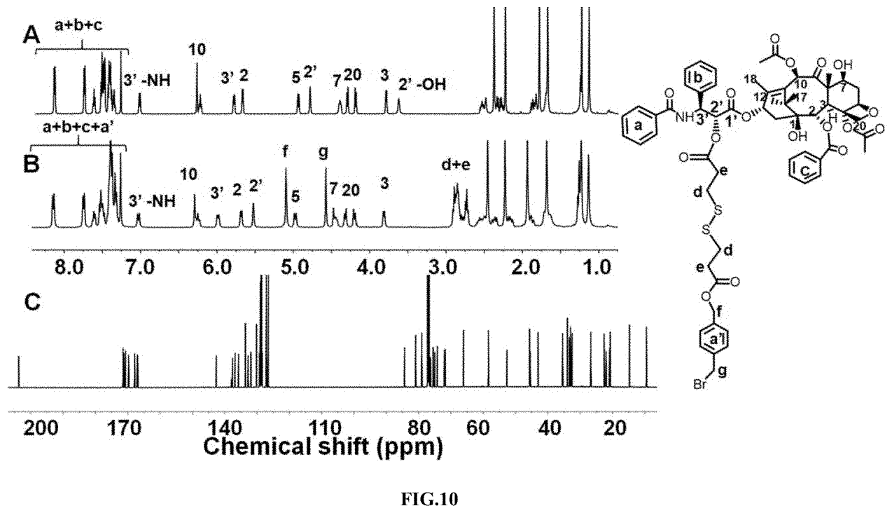

(2) The paclitaxel (500 mg, 1 equivalent) and DTPA-Bz-Br (230 mg, 1 equivalent) were dissolved in dichloromethane, then DMAP (71 mg, 1 equivalent) was added, the mixture was stirred for a few minutes and then dicyclohexylcarbodiimide (DCC, 145 mg, 1.2 equivalents, dissolved in ultra-dry dichloromethane) was added dropwise, after reacting overnight at room temperature, the solvent was evaporated to dryness using a rotary evaporator, and the resulting product was separated by silica gel column chromatography to obtain benzyl bromide structure-modified paclitaxel (PTX-Bz-Br), the eluent was petroleum ether/ethyl acetate system. The NMR spectrum of the product was shown in FIG. 10, the mass spectrum was shown in FIG. 11, and the [M+1]⁺ of m/z was 1229.

2.2 Synthesis of prodrugs of oligonucleotide-paclitaxel conjugate

In this example, a block-type DNA nucleic acid sequence with phosphodiester bonds modified DNA (26 bases) near the 5′end and continuous phosphorothioate bonds modified DNA (19 bases, 18 phosphorothioate bonds) near the 3′end was used.

The nucleic acid sequence is as follows:

^{PO}T₂₆-^{PS}T₁₈-T:
(SEQ ID NO. 5)
5′-TTTTTTTTTTTTTTTTTTTTTTTTTTT*T*T*T*T*T*T*T*T*T*
T*T*T*T*T*T*T*T*T-3′;
(* represents the modification sites of the
phosphorothioate group).
Bcl-2-^{PO}T₁₀-^{PS}T₁₈-T:
(SEQ ID NO. 6)
5′-TCTCCCAGCGTGCGCCATTTTTTTTTTTT*T*T*T*T*T*T*T*T*
T*T*T*T*T*T*T*T*T*T-3;
(* represents the modification sites of the
phosphorothioate group).

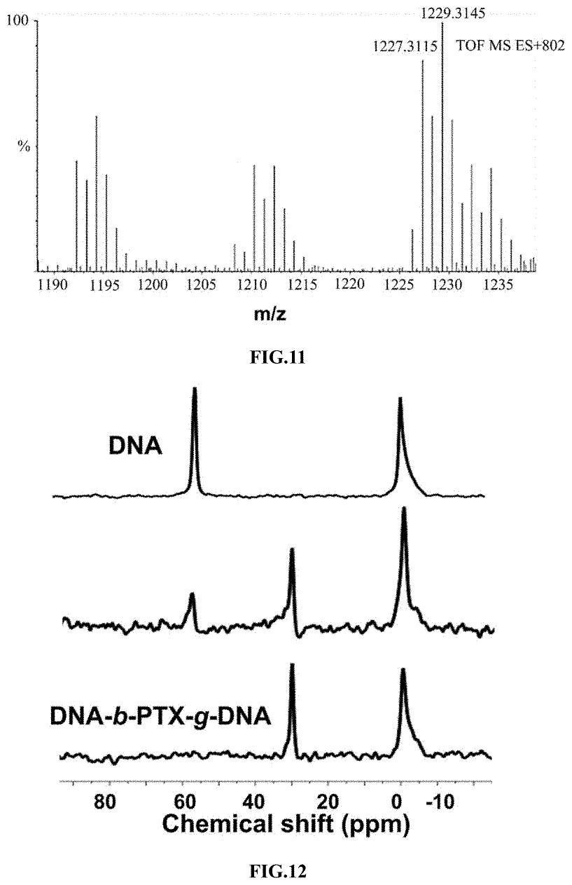

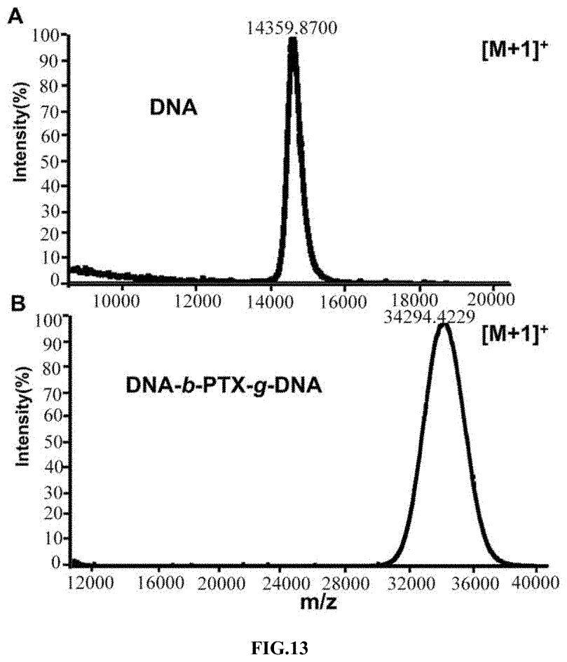

The synthesis method was as follows: PTX-Bz-Br was dissolved in DMSO, DNA was added thereto, then the mixture was shaken to react at 55° C. overnight. After adding water, the excess PTX-Bz-Br in the reaction was removed by extracting with ethyl acetate, the resulting solution was concentrated and evaporated to dryness to obtain the block type nucleic acid-paclitaxel molecule (DNA-b-PTX-g-DNA). The successful grafting was confirmed by NMR spectrum and matrix-assisted laser desorption ionization time of flight mass spectrometry, as shown in FIG. 12 and FIG. 13.

### EXAMPLE 3

### Camptothecin-Conjugated DNA Polyhedron

3.1 Self-assembly of camptothecin-conjugated DNA polyhedrons

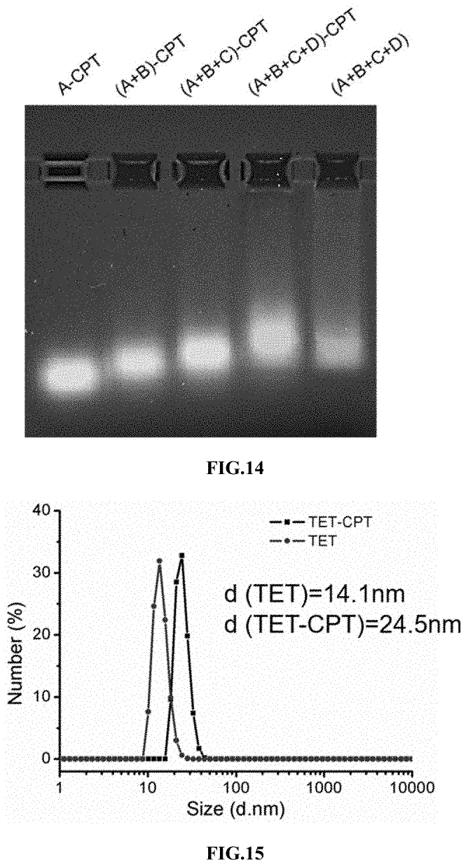

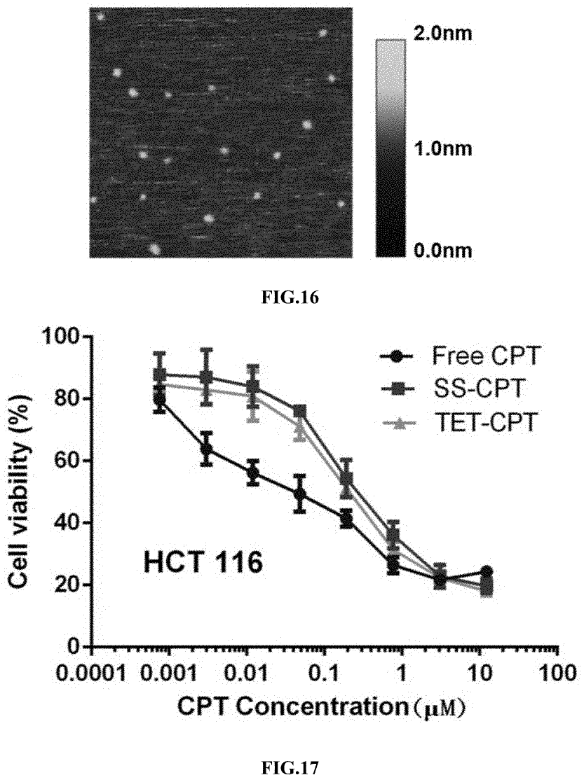

The obtained oligonucleotide prodrug with 4 kinds of base complementary camptothecin-modified were mixed in an equimolar amount in the 1×TAE/Mg²⁺ buffer solution (40 mM Tris, 2 mM ethylenediaminetetraacetate acid disodium salt (EDTA), 20 mM acetic acid, 12.5 mM magnesium acetate tetrahydrate, pH=7.4, the pH value was adjusted by acetic acid), the mixture was heated to 90° C. and rapidly cooled to 4° C. within 2 min. Through the detection of 2% agarose gel electrophoresis, different kinds of oligonucleotide prodrugs are gradually added, the nanostructure became larger and larger, and a polyhedron structure was finally obtained through assembly. Compared with the unmodified DNA polyhedron, the size was increased, as shown in FIG. 14. Dynamic light scattering detection showed that the hydrated particle size of the drug-modified DNA polyhedron was about 24 nm, which was about 10 nm higher than that of the unmodified DNA polyhedron, as shown in FIG. 15. FIG. 16 is a drug-modified DNA polyhedron photographed by an atomic force microscope with a particle size of about 18 nm.

3.2 Anti-tumor effect of camptothecin-conjugated DNA polyhedron in vitro

The DNA polyhedron modified by phosphorothioate-modified oligonucleotides prepared above was able to be effectively taken up by tumor cells to produce cancer cell killing effects similar to or even better than those of the original drug.

After co-incubating the drug-modified polyhedron prepared in Example 3.1 with HCT 116 cells for 72 h, the cell survival rate was detected by MTT. The result was shown in FIG. 17, with the increase of drug concentration, the survival rate of the cells co-incubated with drug-modified polyhedrons decreased gradually, and under higher drug concentrations, the drug-modified polyhedron leads to a lower cell survival rate compared to the original drug, proving that the method of drug delivery by phosphorothioate-modified oligonucleotides was able to achieve effective drug delivery and produce good tumor cell killing effects.

3.3 Apoptosis effect of camptothecin-conjugated DNA polyhedron in vitro

The DNA polyhedron modified by phosphorothioate-modified oligonucleotides of the present disclosure may realize the killing effect on tumor cells by inducing apoptosis of cancer cells.

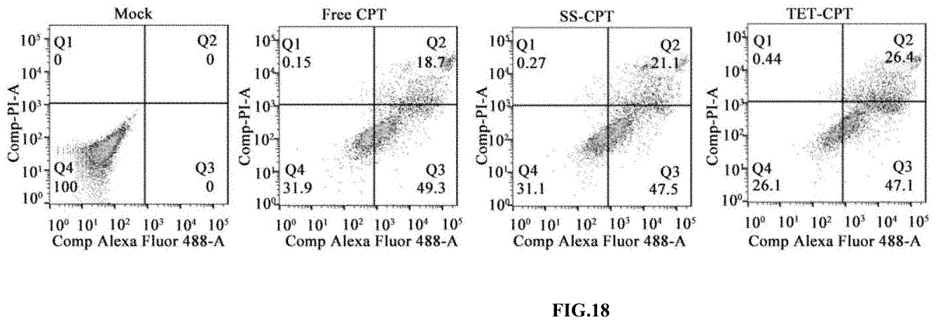

After co-incubating the drug-modified polyhedron prepared in Example 3.1 with HCT 116 cells for 48 h, the tumor cell apoptosis was detected by Annexin V-FITC/PI method, the result was shown in FIG. 18. The cells incubated with drug-modified polyhedrons was able to achieve a good effect of inducing apoptosis of cancer cells, causing an apoptosis rate similar to that of the original drug. It was proved that the method of drug delivery by phosphorothioate-modified oligonucleotides was able to achieve rapid drug release, induce tumor cell apoptosis, and ultimately achieve the purpose of treating cancer.

### EXAMPLE 4

### Micellar Spherical Nucleic Acid Conjugated with Paclitaxel

4.1 Preparation of common nucleic acid block-type paclitaxel-loaded spherical nucleic acid (PTX-SNA)

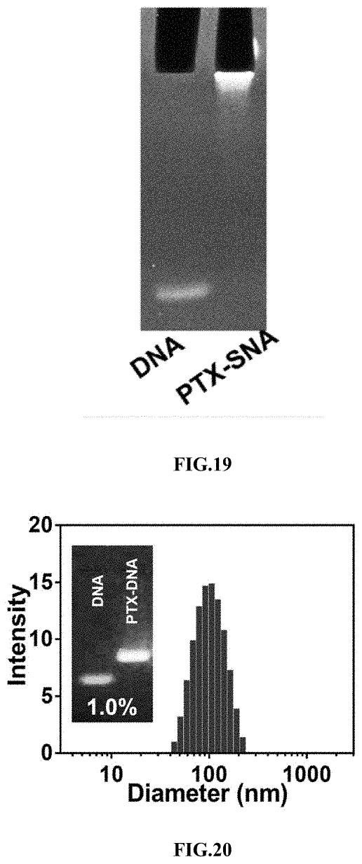

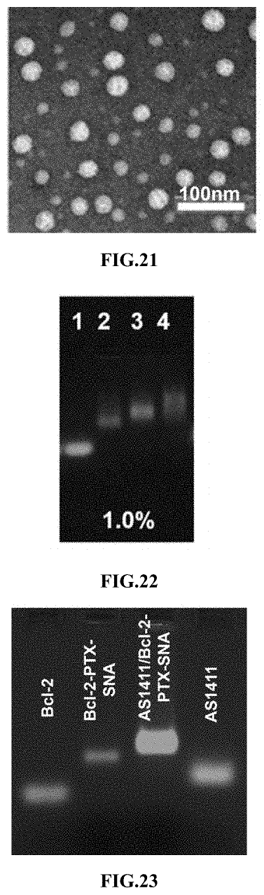

The block-type polyT base sequence (^{PO}T₂₆-^{PS}T₁₈-T) was selected, and the block-type nucleic acid-paclitaxel conjugate (^{PO}DNA-b-(^{PS}DNA-g-PTX)) was obtained according to the method in Example 2.2, the block-type nucleic acid-paclitaxel conjugate was dissolved in DMSO and then an equal volume of water was added, and the solution was dialyzed in the aqueous solution overnight to obtain the PTX-SNA. The successful assembly was confirmed by 20% denaturing polyacrylamide gel electrophoresis and 1% agarose gel electrophoresis. Its particle size and morphology were characterized by dynamic light scattering and transmission electron microscopy. The results are shown in FIG. 19-21.

4.2 Preparation of multifunctional drug-loaded micelles with targeting and imaging functions (AS1411/PTX-SNA, FAM/AS1411/PTX-SNA)

The base sequences of PTX-SNA, polyA₂₀-AS1411 and polyA₂₀-FAM in 4.1 were mixed in a ratio of 1×TAE/Mg²⁺, then the mixture was annealed from 65° C. to room temperature to obtain the multifunctional drug-loaded micelles with targeting and imaging functions. The assembly was characterized by 1% agarose gel, as shown in FIG. 22.

The nucleic acid sequence was as follows:

| | Po1yA20-FAM: |
|---|---|
| (SEQ ID NO. 7) | |
| | 5′-AAAAAAAAAAAAAAAAAAAA-FAM-3′; |
| | Po1yA20_AS1411: |
| (SEQ ID NO. 8) | |
| | 5′-AAAAAAAAAAAAAAAAAAAAGGTGGTGGTGGTTGTGGTGGTGG |
| | TGG-3′. |

4.3 Preparation of non-targeted or targeted drug-loaded spherical nucleic acid co-existing with gene and chemotherapy (Bcl-2-PTX-SNA, AS1411/Bcl-2-PTX-SNA). By changing the phosphodiester bond sequence in the block-type DNA to the antisense nucleic acid Bcl-2 sequence, the purpose of reversing the tumor multidrug resistance can be achieved.

The functional nucleic acid (antisense nucleic acid) was selected as block Bcl-2-^{PO}T₁₀-^{PS}T₁₈-T base sequence, the block-type nucleic acid-paclitaxel conjugate containing functional nucleic acid sequence (Bcl-2-b-(^{PS}DNA-g-PTX)) was obtained according to the method in Example 2.2, the obtained conjugate was dissolved in DMSO and an equal volume of water was added, the solution was dialyzed overnight in an aqueous solution to obtain the non-targeted Bcl-2-PTX-SNA co-existing with genes and chemotherapy. The SNA obtained by co-dialysis of Bcl-2-b-(^{PS}DNA-g-PTX) and ^{PO}T₂₆-b-(^{PS}DNA-g-PTX) in proportion was mixed with polyA₂₀-AS1411 in proportion in 1×TAE/Mg²⁺, and the targeted AS1411/Bcl-2-PTX-SNA co-existing with genes and chemotherapy was obtained by annealing from 65° C. to room temperature. The assembly was characterized by 0.5% agarose gel, as shown in FIG. 23.

4.4 In vitro antitumor and reversal of tumor multidrug resistance of micellar spherical nucleic acid conjugated with paclitaxel

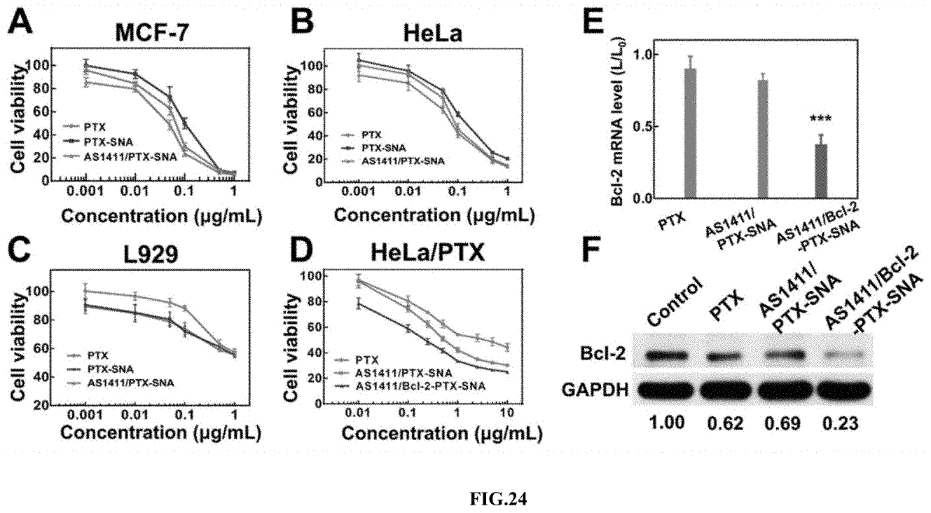

After co-incubating the various PTX-SNAs prepared in Examples 4.1-4.3 with tumor cells for 72 h, the cell survival rate was detected by MTT, and the results were shown in FIG. 24. Among them, MCF-7 and HeLa cells are sensitive tumor cells, and L929 cells are normal cells, which are used to characterize the anti-tumor effect of the targeted AS1411/PTX-SNA; HeLa/PTX are paclitaxel-resistant cells used to characterize the effect of AS1411/Bcl-2-PTX-SNA in reversing tumor resistance. The experimental results showed that AS1411/PTX-SNA modified by targeting molecules had a better targeting function on tumor cells, and the tumor cell killing effect is better than that of non-targeted PTX-SNA. For the targeted AS1411/Bcl-2-PTX-SNA co-existing with gene and chemotherapy, the purpose of reversing multi-drug resistance of tumors can be achieved by down-regulating the gene expression of anti-apoptotic Bcl-2 protein, achieving the purpose of a good combined treatment of gene and chemotherapy.

In the above example of the present disclosure, the DNA tetrahedral origami structure of the drug-loaded system was firstly obtained, and the hydrated nanometer particle size was 20 nanometers. The extremely hydrophobic camptothecin drug molecule was accurately grafted into the oligonucleotide chain, so that the solubility of the camptothecin drug was improved, and forms a water-soluble nano-prodrug, and the assembly of the polyhedral structure was realized through a pre-designed DNA sequence, achieving the structural controllability of the drug-loaded system. Secondly, in the above embodiments of the present disclosure, drug-loaded micellar spherical nucleic acids with a hydrated particle size of 68 nanometers were also obtained; the paclitaxel drug is accurately grafted onto the phosphorothioate group of the nucleic acid, a micelle structure with the drug as the core and the nucleic acid block of the phosphodiester bond as the outer shell is prepared by using the block-type DNA structure of phosphodiester bond and phosphorothioate bond and the hydrophobic property of paclitaxel. The drug loading amount of the prepared micelle was up to 53%.

Under the teaching of the present disclosure and the above examples, those skilled in the art can easily foresee that the raw materials or their equivalent substitutes, processing methods or their equivalent substitutes listed or exemplified in the present disclosure can realize the present disclosure. As well as the upper and lower limits and interval values of the parameters of the raw materials and processing methods, the present disclosure can be realized, and the embodiments are not listed here.

1. A nucleic acid-drug conjugate based on a phosphorothioate-modified nucleic acid, wherein comprising a phosphorothioate-modified nucleic acid skeleton and a drug molecule grafted onto the nucleic acid skeleton, and the grafting is realized by reacting the phosphorothioate groups on the nucleic acid skeleton with a group modified on the drug molecule that able to undergo electrophilic reaction with the phosphorothioate groups.

2. The nucleic acid-drug conjugate according to claim 1, wherein on the phosphorothioate-modified nucleic acid skeleton, the sites and number of phosphorothioate modifications are able to be adjusted and controlled as required, the phosphorothioate group is continuously modified at one end of the nucleic acid sequence, and/or is selectively modified at the middle base sequence of the nucleic acid sequence, and the modification mode is multiple modification or single modification.

3. The nucleic acid-drug conjugate according to claim 1, wherein the phosphorothioate-modified nucleic acid skeleton is prepared by a solid phase synthesis method.

4. The nucleic acid-drug conjugate according to claim 1, wherein the sequence and segment type of the oligonucleotide of the phosphorothioate-modified nucleic acid skeleton are able to be independently designed, and a controllable DNA nanostructure is able to be further assembled through molecular recognition, and the nucleic acid-drug conjugate and the assembled structure thereof is able to be used as a novel drug delivery system.

5. The nucleic acid-drug conjugate according to claim 2, wherein a phosphorothioate modification site is set every 2 to 3 bases on the nucleic acid skeleton of the nucleic acid used for the assembly of gel and tetrahedral structure; continuous phosphorothioate modifications at one end of a nucleic acid sequence are carried out on the nucleic acid used for micellar assembly to prepare block-type nucleic acids containing phosphodiester bonds and phosphorothioate bonds.

6. The nucleic acid-drug conjugate according to claim 1, wherein a group that is able to undergo electrophilic reaction with phosphorothioate group is introduced into the drug molecule by a simple esterification or acylation reaction.

7. The nucleic acid-drug conjugate according to claim 1, wherein a cleavable responsive chemical bond is further introduced into the drug molecule.

8. The nucleic acid-drug conjugate according to claim 1, wherein the group that is able to modified on the drug molecule and undergo electrophilic reaction with phosphorothioate group is selected from one or more of the following: 1) a bromine-containing or iodine-containing functional group; 2) a maleimide group; 3) an aziridinyl sulfonamide group.

9. The nucleic acid-drug conjugate according to claim 1, wherein the drug molecule is selected from one or more of anticancer drugs or cancer targeted drug molecules.

10. The nucleic acid-drug conjugate according to claim 1, wherein the drug molecule grafted on the nucleic acid skeleton is a functional drug molecule, a fluorescent probe molecule, or a cell targeted molecule.

11. The nucleic acid-drug conjugate according to claim 1, wherein the type of the nucleic acid skeleton is selected from a deoxyribonucleic acid sequence or a ribonucleic acid sequence; the sequence of the nucleic acid skeleton is selected one or more of:
a non-functional common base sequence, including a simple nucleic acid sequence composed of one base and a complex nucleic acid sequence that is able to be used for precise structural assembly of nucleic acids;
a functional nucleic acid sequence, the functional nucleic acid sequence is selected from antisense nucleic acid sequence, nucleic acid aptamer sequence, nuclease sequence, small interfering RNA, messenger RNA, microRNA, long non-coding RNA, small hairpin RNA, guide RNA for gene editing, and circular RNA.

12. The nucleic acid-drug conjugate according to claim 1, wherein the nucleic acid molecule grafted with the drug retains the property of base complementary pairing, by which the other functional nucleic acid sequences are paired to impart targeting and imaging functions to the nucleic acid-drug conjugate drug delivery system to prepare a multifunctional drug delivery system of nucleic acid-drug conjugate, wherein the functional nucleic acid for pairing is selected from nucleic acid aptamers, antisense nucleic acid sequences, fluorescent molecule-modified nucleic acid sequences, functional polypeptide-modified nucleic acid sequences, and targeted galactose-modified nucleic acid sequences.

13. A drug delivery system, wherein the drug delivery system is a nano drug-loaded system formed by self-assembly of the nucleic acid-drug conjugate according to claim 1.

14. The drug delivery system according to claim 13, wherein different drug delivery systems are able to be prepared by different methods according to the selected drug molecules and nucleic acid sequences, and the drug delivery systems are selected from simple chain nucleic acid-drug macromolecule drugs, precisely assembled DNA nanostructures, DNA nanogels, drug-loaded micellar spherical nucleic acid nanostructures, drug-loaded nucleic acid polyhedral structures, and drug-loaded nucleic acid hydrogels.

15. The drug delivery system according to claim 14, wherein the drug molecules used to prepare the precisely assembled DNA nanostructures are selected from molecules with weak hydrophobicity and small molecular weight, and/or molecules with reduced number of phosphorothioate modifications on the nucleic acid skeleton; and the drug molecules used for micellar assembly are selected from highly hydrophobic molecules, and/or molecules with increased number of phosphorothioate modifications on the nucleic acid skeleton.

16. A method for preparing the nucleic acid-drug conjugate according to claim 1, wherein comprising:
step one, preparing a phosphorothioate-modified nucleic acid molecule;
preparing a drug molecule containing a group that is able to undergo electrophilic reaction with phosphorothioate group by a chemical reaction method;
there is no order among the steps of preparing the nucleic acid molecule and preparing the drug molecule;
step two, preparing a nucleic acid-drug conjugate, dissolving the modified drug molecule in an organic solvent, then adding an appropriate amount of nucleic acid molecule for the reaction, the drug molecule in the reaction is greatly excessive relative to the phosphorothioate groups, removing the excess small molecules after reaction, and drying to obtain the nucleic acid-drug conjugate.

17. The method for preparing the nucleic acid-drug conjugate according to claim 16, wherein the phosphorothioate-modified nucleic acid molecule is prepared by a solid phase synthesis.

18. The method for preparing the nucleic acid-drug conjugate according to claim 16, wherein the modification method of the drug molecule is selected from the following methods: (1) preparing carbonyl ethyl bromide drug molecules containing disulfide bonds through a two-step esterification reaction; and (2) preparing benzyl bromide structure-modified drug molecules containing disulfide bonds through a two-step esterification reaction.

19. The method for preparing the nucleic acid-drug conjugate according to claim 16, wherein the drug molecule is further introduced with a cleavable responsive chemical bond, the cleavable responsive chemical bond is a disulfide bond, and the group that undergo electrophilic reaction with phosphorothioate groups is a bromo-modified group; the chemical reaction method for preparing a bromo-modified drug molecule containing disulfide bonds is selected from the following methods: introducing the disulfide bond into the camptothecin drug molecule through triphosgene under argon atmosphere, and then reacting with bromoacetyl bromide to obtain the camptothecin bromide drug molecule; and, carrying out an esterification reaction of 4-bromomethylbenzyl alcohol with dithiodipropylene to prepare the carboxylic acid structure containing disulfide bonds, and then carrying out an esterification reaction of the carboxylic acid with the 2′ hydroxyl group of paclitaxel to prepare the benzyl bromide structure-modified paclitaxel drug molecule.

20. The method for preparing the nucleic acid-drug conjugate according to claim 16, wherein the grafting efficiency of drug molecules is able to be controlled by controlling the concentration of nucleic acid molecules, the ratio of drug molecules to phosphorothioate groups, and whether a salt solution is included in the reaction solution.

21-24. (canceled)


SEQUENCE LISTING (8 sequences)

SEQ ID NO: 1 - DNA, 55 bp, Artificial Sequence
  sequence of the oligonucleotide TET-A
acattcctaagtctgaaacattacagcttgctacacgagaagagccgccatagta

SEQ ID NO: 2 - DNA, 55 bp, Artificial Sequence
  sequence of the oligonucleotide TET-B
tatcaccaggcagttgacagtgtagcaagctgtaatagatgcgagggtccaatac

SEQ ID NO: 3 - DNA, 46 bp, Artificial Sequence
  sequence of the oligonucleotide TET-C
tcaactgcctggtgataaaacgacactacgactatggcggctcttc

SEQ ID NO: 4 - DNA, 55 bp, Artificial Sequence
  sequence of the oligonucleotide TET-D
ttcagacttaggaatgtgcttcccacgtagtgtcgtttgtattggaccctcgcat

SEQ ID NO: 5 - DNA, 45 bp, Artificial Sequence
  nucleic acid sequence POT26-PST18-T
ttttttttttttttttttttttttttttttttttttttttttttt

SEQ ID NO: 6 - DNA, 47 bp, Artificial Sequence
  nucleic acid sequence Bcl-2-POT10-PST18-T
tctcccagcgtgcgccatttttttttttttttttttttttttttttt

SEQ ID NO: 7 - DNA, 20 bp, Artificial Sequence
  nucleic acid sequence PolyA20-FAM
aaaaaaaaaaaaaaaaaaaa

SEQ ID NO: 8 - DNA, 46 bp, Artificial Sequence
  nucleic acid sequence PolyA20-AS1411
aaaaaaaaaaaaaaaaaaaaggtggtggtggttgtggtggtggtgg